(19) United States
(12) Patent Application Publication
(10) Pub. No.: US 2016/0013389 A1
(43) Pub. Date: Jan. 14, 2016

(54) THERMOCHEMICAL GAS SENSOR USING CHALCOGENIDE-BASED NANOWIRES AND METHOD FOR MANUFACTURING THE SAME

(71) Applicant: INDUSTRY-UNIVERSITY COOPERATION FOUNDATION HANYANG UNIVERSITY ERICA CAMPUS, Gyeonggi-do (KR)

(72) Inventors: Yong Ho Choa, Gyeonggi-do (KR); Seil Kim, Gyeonggi-do (KR); Young In Lee, Gyeonggi-do (KR); Yo Min Choi, Gyeonggi-do (KR)

(21) Appl. No.: 14/770,921

(22) PCT Filed: Feb. 26, 2014
(86) PCT No.: PCT/KR2014/001548

§ 371 Date: Aug. 27, 2015

(30) Foreign Application Priority Data
Feb. 27, 2013 (KR) 10-2013-0020889

(51) Int. Cl. H01L 35/16; G01N 33/00; H01L 35/34; G01N 27/12; H01L 35/04; H01L 35/32

(52) U.S. Cl. CPC H01L 35/16; H01L 35/04; H01L 35/32; H01L 35/34; G01N 27/125; G01N 33/005

(57) ABSTRACT

The present invention relates to a thermochemical gas sensor using chalcogenide-based nanowires and a method for same, comprising: a porous alumina template comprising a front surface, a rear surface, and side surfaces and provided with a plurality of pores which penetrate the front surface and the rear surface; a seed layer provided on the rear surface of the porous alumina template for covering the plurality of pores and having electric conductivity; a plurality of chalcogenide-based nanowires provided inside the plurality of pores and coming into contact with the seed layer, which is exposed through the plurality of pores; an electrode provided on the front surface of the porous alumina template and coming into contact with the chalcogenide-based nanowires; an electrode wire for electrically connecting with the electrode; and a porous white gold-alumina composite or a porous palladium-alumina composite provided above the electrode for causing a heat-emitting reaction by coming into contact with a gas to be detected, wherein the chalcogenide-based nanowires comprise BiₓTe_{y}(1.5≦x≦2.5, 2.4≦y≦3.6), SbₓTe_{y}(1.5≦x≦2.5, 2.4≦y≦3.6) or (Bi₁₋ₓSbₓ)Te₃(0<x<1). According to the present invention, a variety of gases can be detected through a change in the porous white gold-alumina composite or the porous palladium-alumina composite, and temperature and minute changes in electromotive force can be confirmed by detecting the gases, and thus the present invention can be utilized for evaluating a thermochemistry performance by using gas.

### TECHNICAL FIELD

The present invention relates to a thermochemical gas sensor and a method of manufacturing the same, and more particularly, to a thermochemical gas sensor which can sense a variety of desirable gases by changes in a porous platinum-alumina composite or porous palladium-alumina composite in response to a gas to be sensed and thereby detect changes in temperatures and subtle changes in electromotive force in accordance with a principle of generating an electromotive force by a temperature change, and thus can be utilized to evaluate a thermoelectric figure of merit using a gas, and a method of manufacturing the same.

### BACKGROUND ART

Although a hydrogen gas has been in the limelight as a future clean fuel, due to characteristic properties of the hydrogen gas, it has to be more precisely and completely sensed than other combustible gases.

Generally, since the hydrogen gas has a wide range of explosion concentrations from 4 to 75%, for practical supply and use, a sensor has to be qualified for possibility of sensing at low concentrations and a wide range of gas concentrations, no influence on other gases, except the hydrogen gas, a vapor (including a humidity) or a temperature, high sensing accuracy and miniaturization. There is a variety of research on various types of hydrogen sensors having the above-described characteristics. Types of hydrogen sensors that are concentrating upon the research today are classified into two groups: one group includes a contact combustion-type hydrogen sensor, a hot wire-type hydrogen sensor, and a thermoelectronic hydrogen sensor; and the other group includes a semiconductor-type hydrogen sensor, an electrochemical hydrogen sensor, and a metal absorption-type hydrogen sensor, which employ a property of changing a resistance due to the change in electron density on a particle surface, when hydrogen is attached.

The top priority in hydrogen sensing is possibility of sensing hydrogen at room temperature, and in further manufacturing of a device, in order to ensure price competitiveness, it is necessary to develop technology of synthesizing a material at room temperature, instead of a high vacuum and high temperature process, which needs a high processing cost.

Since a SiGe-based thin film hydrogen sensor has a high Seebeck coefficient of SiGe at a high temperature, for practical use as a sensor, it has to be operated at a high temperature using a platinum-heater (Pt-heater). A palladium-based hydrogen sensor, which is used as a representative hydrogen sensor, uses high-priced palladium nanoparticles and nanowires and requires a high temperature and a high vacuum in material and sensor manufacturing processes, and thus it is difficult to manufacture a low-priced sensor.

Most of the research is focused on a palladium/platinum gate field effect transistor (FET) type, but because of the degradation in sensing capability in a high concentration region and degradation in performance caused by a drastic phase change when a palladium-based sensor is repeatedly exposed to a hydrogen gas, more research on a sensor that can sense a wide range of concentrations of the hydrogen gas is needed.

In addition, with the development of and an increased demand for a hydrogen fuel cell, which is being in the limelight as future clean energy, in the field of automobiles, research on producing an energy source using waste heat, which is made of a thermoelectric material, is needed as well as ensuring of stability to a fuel cell, and since a hydrogen battery is also used in the field of aerospace technology, for example, a satellite, a space shuttle, etc., the development of a suitable hydrogen sensor for the above purpose is needed. Also, it is necessary to study methods of mass-producing a compact and high-sensitive hydrogen sensor in accordance with micro electro mechanical systems (MEMS), which is one of the microscopic circuit manufacturing techniques.

### DISCLOSURE

### Technical Problem

It is one object of the present invention to provide a thermochemical gas sensor which can sense a variety of desirable gases by changes in a porous platinum-alumina composite or porous palladium-alumina composite in response to a gas to be sensed and thereby detect changes in temperatures and subtle changes in electromotive force in accordance with a principle of generating an electromotive force by a temperature change, and thus can be utilized to evaluate a thermoelectric figure of merit using a gas.

It is another object of the present invention to provide a method of manufacturing a thermochemical gas sensor which uses electrodeposition employing a low-priced synthesis method, thereby obtaining a sensor at room temperature without using a high vacuum and high temperature process which needs a high process cost, and can minimize the amount of a material applied to each device, and therefore ensure price competitiveness.

### Technical Solution

The present invention provides a thermochemical gas sensor, which includes: a porous alumina template having top, bottom and side surfaces and including a plurality of pores penetrating the top and bottom surfaces; a seed layer with electric conductivity formed on the bottom surface of the porous alumina template to cover the plurality of pores; a plurality of chalcogenide-based nanowires, which is in contact with the seed layer exposed through the plurality of pores and formed in the plurality of pores; an electrode, which is in contact with the chalcogenide-based nanowires and formed on the top surface of the porous alumina template; electrode wires electrically connected with the electrode; and a porous platinum-alumina composite or porous palladium-alumina composite, which is formed above the electrode and causes an exothermic reaction when in contact with a gas to be sensed, wherein the chalcogenide-based nanowires consist of BiₓTe_{y} (1.5≦x≦2.5, 2.4≦y≦3.6), SbₓTe_{y} (1.5≦x≦2.5, 2.4≦y≦3.6) or (Bi₁₋ₓSbₓ)Te₃ (0<x<1).

The seed layer may have a thickness of 10 to 1000 nm, and consist of at least one metal selected from gold (Au), silver (Ag) and copper (Cu).

The pores may have an average diameter of 10 to 1000 nm, and the chalcogenide-based nanowires may have an average diameter of 1 to 500 nm smaller than that of the pores.

The length of the chalcogenide-based nanowires is the same as or smaller than the depth of the pores, and the porous platinum-alumina composite or porous palladium-alumina composite may be a porous material having a plurality of macropores and a plurality of mesopores.

In addition, the present invention provides a thermochemical gas sensor, which includes: a porous alumina template having top, bottom and side surfaces and including a plurality of pores penetrating the top and bottom surfaces; a seed layer with electric conductivity, which is formed on the bottom surface of the porous alumina template to cover the plurality of pores; a plurality of P-type chalcogenide-based nanowires, which is in contact with the seed layer exposed through the plurality of pores and formed in the plurality of pores; a plurality of N-type chalcogenide-based nanowires, which is in contact with the seed layer exposed through the plurality of pores and formed in the plurality of pores; an electrode, which is in contact with the P-type chalcogenide-based nanowires and the N-type chalcogenide-based nanowires and formed on the top surface of the porous alumina template; electrode wires electrically connected with the electrode; and a porous platinum-alumina composite or porous palladium-alumina composite, which is formed above the electrode and causes an exothermic reaction when in contact with a gas to be sensed, wherein the P-type chalcogenide-based nanowires consist of SbₓTe_{y} (1.5≦x≦2.5, 2.4≦y≦3.6) or (Bi₁₋ₓSbₓ)Te₃ (0<x<1), and the N-type chalcogenide-based nanowires consist of BiₓTe_{y} (1.5≦x≦2.5, 2.4≦y≦3.6).

The seed layer may have a thickness of 10 to 1000 nm, and consist of at least one metal selected from gold (Au), silver (Ag) and copper (Cu).

The pores may have an average diameter of 10 to 1000 nm, and the chalcogenide-based nanowires may have an average diameter of 1 to 500 nm smaller than that of the pores.

The length of the chalcogenide-based nanowires is the same as or smaller than the depth of the pores, and the porous platinum-alumina composite or porous palladium-alumina composite may be a porous material having a plurality of macropores and a plurality of mesopores.

In addition, the present invention provides a method of manufacturing a thermochemical gas sensor, the method includes: preparing a porous alumina template having top, bottom and side surfaces and including a plurality of pores penetrating the top and bottom surfaces; forming a seed layer with electric conductivity on the bottom surface of the porous alumina template to cover the plurality of pores; growing and forming a plurality of chalcogenide-based nanowires on the seed layer exposed through the plurality of pores using electrodeposition; forming an electrode in contact with the chalcogenide-based nanowires on the top surface of the porous alumina template; forming electrode wires electrically connected with the electrode; and forming a porous platinum-alumina composite or porous palladium-alumina composite above the electrode formed on the top surface of the porous alumina template, the composite causing an exothermic reaction when in contact with a gas to be sensed, wherein the chalcogenide-based nanowires consist of BiₓTe_{y} (1.5≦x≦2.5, 2.4≦y≦3.6), SbₓTe_{y} (1.5≦x≦2.5, 2.4≦y≦3.6) or (Bi₁₋ₓSbₓ)Te₃ (0<x<1), the electrodeposition uses an electrolyte, which contains at least one material selected from a bismuth (Bi) precursor and an antimony (Sb) precursor; a tellurium (Te) precursor; and an acid, and the acid is a material that can dissolve at least one selected from the bismuth (Bi) precursor and the antimony (Sb) precursor, and the tellurium (Te) precursor.

The bismuth (Bi) precursor may be Bi(NO₃)₃.5H₂O, the antimony (Sb) precursor may be Sb₂O₃, the tellurium (Te) precursor may be TeO₂, and the acid may be HNO₃.

When the chalcogenide-based nanowires consist of SbₓTe_{y} (1.5≦x≦2.5, 2.4≦y≦3.6) or (Bi₁₋ₓSbₓ)Te₃ (0<x<1), prior to the electrode formation after the growth of the chalcogenide-based nanowires, annealing may be performed on the chalcogenide-based nanowires at 100 to 300° C.

The seed layer may have a thickness of 10 to 1000 nm, and consist of at least one metal selected from gold (Au), silver (Ag) and copper (Cu).

The electrode may be formed by electroplating at least one metal selected from gold (Au), silver (Ag) and copper (Cu), and the electroplating may be performed by applying a current to a two-electrode system using a rectifier, while stirring with a magnetic bar.

The pores may have an average diameter of 10 to 1000 nm, the chalcogenide-based nanowires may have an average diameter of 1 to 500 nm smaller than that of the pores, and the length of the chalcogenide-based nanowires may be formed the same as or smaller than the depth of the pores.

The porous platinum-alumina composite or porous palladium-alumina composite may be formed by preparing a mixed solution of styrene and distilled water, synthesizing a polystyrene solution by adding potassium persulfate to the mixed solution, drying the polystyrene solution to obtain colloidal crystals, synthesizing a precursor solution of the platinum-alumina composite or palladium-alumina composite, immersing the colloidal crystals obtained by drying in the precursor solution of the platinum-alumina composite or palladium-alumina composite, and drying and calcining the colloidal crystals immersed in the precursor solution of the platinum-alumina composite or palladium-alumina composite to remove the polystyrene colloidal crystals, wherein the porous platinum-alumina composite or porous palladium-alumina composite may be formed to have a plurality of macropores and a plurality of mesopores.

In addition, the present invention provides a method of manufacturing a thermochemical gas sensor, the method including: preparing a porous alumina template having top, bottom and side surfaces and including a plurality of pores penetrating the top and bottom surfaces, masking regions of the bottom surface of the porous alumina template, except the part in which chalcogenide-based nanowires are to be formed, and forming a seed layer with electric conductivity on the exposed part to cover a plurality of pores; covering a region in which N-type chalcogenide-based nanowires are to be formed on the top surface of the porous alumina template with a first mask, and growing and forming a plurality of P-type chalcogenide-based nanowires on the seed layer exposed through the plurality of pores using electrodeposition; covering the region in which the P-type chalcogenide-based nanowires have been formed on the top surface of the porous alumina template with a second mask, and growing and forming a plurality of N-type chalcogenide-based nanowires on the seed layer exposed through the plurality of pores by removal of the first mask using electrodeposition; forming an electrode in contact with the P-type chalcogenide-based nanowires and the N-type chalcogenide-based nanowires on the top surface of the porous alumina template; forming electrode wires electrically connected with the electrode; and forming a porous platinum-alumina composite or porous palladium-alumina composite above the electrode formed on the top surface of the porous alumina template, the composite causing an exothermic reaction when in contact with a gas to be sensed, wherein the P-type chalcogenide-based nanowires consist of SbₓTe_{y} (1.5≦x≦2.5, 2.4≦y≦3.6) or (Bi₁₋ₓSbₓ)Te₃ (0<x<1), the N-type chalcogenide-based nanowires consist of BiₓTe_{y} (1.5≦x≦2.5, 2.4≦y≦3.6), the electrodeposition for forming the P-type chalcogenide-based nanowires uses an electrolyte containing one or both of an antimony (Sb) precursor and a bismuth (Bi) precursor, a tellurium (Te) precursor and an acid, the electrodeposition for forming the N-type chalcogenide-based nanowires uses an electrolyte containing a bismuth (Bi) precursor, a tellurium (Te) precursor and an acid, and the acid is a material that can dissolve an antimony (Sb) precursor, a bismuth (Bi) precursor and a tellurium (Te) precursor.

The bismuth (Bi) precursor may be Bi(NO₃)₃.5H₂O, the antimony (Sb) precursor may be Sb₂O₃, the tellurium (Te) precursor may be TeO₂, and the acid may be HNO₃.

When the chalcogenide-based nanowires consist of SbₓTe_{y} (1.5≦x≦2.5, 2.4≦y≦3.6) or (Bi₁₋ₓSbₓ)Te₃ (0<x<1), prior to the electrode formation after the growth of the chalcogenide-based nanowires, annealing may be performed on the chalcogenide-based nanowires at 100 to 300° C.

The seed layer may have a thickness of 10 to 1000 nm, and consist of at least one metal selected from gold (Au), silver (Ag) and copper (Cu).

The electrode may be formed by electroplating at least one metal selected from gold (Au), silver (Ag) and copper (Cu), and the electroplating may be performed by applying a current to a two-electrode system using a rectifier, under stirring with a magnetic bar.

The pores may have an average diameter of 10 to 1000 nm, the chalcogenide-based nanowires may have an average diameter of 1 to 500 nm smaller than that of the pores, and the length of the chalcogenide-based nanowires may be formed the same as or smaller than the depth of the pores.

The porous platinum-alumina composite or porous palladium-alumina composite may be formed by preparing a mixed solution of styrene and distilled water, synthesizing a polystyrene solution by adding potassium persulfate to the mixed solution, drying the polystyrene solution to obtain colloidal crystals, synthesizing a precursor solution of the platinum-alumina composite or palladium-alumina composite, immersing the colloidal crystals formed by drying in the precursor solution of the platinum-alumina composite or palladium-alumina composite, and drying and calcining the colloidal crystals immersed in the precursor solution of the platinum-alumina composite or palladium-alumina composite to remove the polystyrene colloidal crystals, wherein the porous platinum-alumina composite or porous palladium-alumina composite may be formed to have a plurality of macropores and a plurality of mesopores.

### Advantageous Effects

A thermochemical gas sensor according to the present invention is manufactured by forming a single thermoelectric device or a P-N junction thermoelectric device having maximized thermoelectric properties by selectively plating a chalcogenide-based nanowires known as a thermoelectric material in a porous alumina template using electrodeposition, and binding a porous catalyst-alumina composite causing an exothermic reaction when in contact with a gas to be sensed, and the thermochemical gas sensor is a new thermoelectric nanowire array-based thermochemical gas sensor that can serve to sense a gas and evaluate a gas sensing property.

The thermochemical gas sensor of the present invention can also be used as a thermoelectric hydrogen gas sensor to which chalcogenide-based nanowires having a large specific surface area, and characteristic electrical and optical properties are applied.

BiₓTe_{y} (1.5≦x≦2.5, 2.4≦y≦3.6), SbₓTe_{y} (1.5≦x≦2.5, 2.4≦y≦3.6) or (Bi₁₋ₓSbₓ)Te₃ (0<x<1) for forming the chalcogenide-based nanowires is a material that exhibits high thermoelectric properties at room temperature, and can be easily synthesized using electrodeposition. By the electrodeposition, thermoelectric materials exhibiting thermoelectric properties in a temperature range suitable for an operating temperature can be easily synthesized.

According to the present invention, the principle in which an electromotive force is generated according to the change in temperature is used, and a variety of desired types of gases can be sensed according to the change in a porous platinum-alumina composite or porous palladium-alumina composite in response to a gas to be sensed (e.g., a hydrogen gas). In addition, a temperature change and the subtle changes in electromotive force can be detected by sensing the gas, and therefore the gas sensor of the present invention can also be used in the evaluation of a thermoelectric figure of merit using a gas.

A method of manufacturing a thermochemical gas sensor according to the present invention uses electrodeposition employing a low-priced synthesis method, thereby obtaining a sensor at room temperature without using a high vacuum and high temperature process which needs a high process cost, and can minimize the amount of a material applied to each device, and therefore ensure price competitiveness.

Also, with the development of and an increased demand for hydrogen fuel cells, which are being in the limelight as future clean energy, it is considered that, in the automotive field, stability to the fuel cells can be ensured, and an energy source can be produced from a thermoelectric material using waste heat.

Also, since a hydrogen battery is also used in the field of aerospace technology, for example, a satellite, a space shuttle, etc., the development of a suitable hydrogen sensor for the above purpose is needed, and it is necessary to study methods of mass-producing a compact and high-sensitive hydrogen sensor in accordance with micro electro mechanical systems (MEMS), which is one of the microscopic circuit manufacturing technology. Moreover, it is considered that the gas sensor of the present invention can be applied to MEMS technology through the downsizing of the thermochemical gas sensor manufactured according to the present invention and the development of integrated application/coating technology of a catalyst using inkjet printing.

### DESCRIPTION OF DRAWINGS

FIGS. 1 to 4 are schematic diagrams illustrating a process of manufacturing a thermochemical gas sensor using a single thermoelectric device according to a first exemplary embodiment of the present invention.

FIGS. 5 to 10 are schematic drawings illustrating a process of manufacturing a thermochemical gas sensor using a P-N junction thermoelectric device according to a second exemplary embodiment of the present invention.

FIG. 11 is an optical microscope image of a cross-section of a porous alumina template that is transversely cut after BiₓTe_{y} nanowires are formed in the porous alumina template using electrodeposition according to Example 1.

FIG. 12 is a graph representing the lengths of the BiₓTe_{y} nanowires as a function of plating time, wherein the BiₓTe_{y} nanowires are synthesized in the porous alumina template through electroplating according to Example 1.

FIG. 13 is an optical microscope image of a cross-section of a porous alumina template that is transversely cut after SbₓTe_{y} nanowires are synthesized in the porous alumina template through electroplating according to Example 2.

FIG. 14 is a graph representing the lengths of the SbₓTe_{y} nanowires as a function of plating time, wherein the SbₓTe_{y} nanowires are synthesized in the porous alumina template through electroplating according to Example 2.

FIGS. 15 and 16 are graphs representing the X-ray diffraction (XRD) results of the BiₓTe_{y} nanowires synthesized by electroplating according to Example 1.

FIG. 17 is a graph representing the XRD results of the SbₓTe_{y} nanowires synthesized by electroplating according to Example 2.

FIG. 18 shows the FE-SEM image and energy dispersive spectroscopy (EDS) analysis result for the BiₓTe_{y} nanowires synthesized by electroplating according to Example 1.

FIG. 19 shows the FE-SEM images and energy dispersive spectroscopy (EDS) analysis results, obtained before and after the annealing, for the SbₓTe_{y} nanowires synthesized by electroplating according to Example 2.

FIG. 20 is a graph representing the changes in temperature of the porous platinum-alumina composite plotted with respect to hydrogen concentrations when hydrogen sensing takes place in a thermochemical gas sensor to which a single thermoelectric device composed of the BiₓTe_{y} nanowires is applied according to Example 1, and FIG. 21 is a graph representing the changes in electromotive force occurring in the thermoelectric device plotted with respect to hydrogen concentrations when the hydrogen sensing takes place in the thermochemical gas sensor to which the single thermoelectric device composed of the BiₓTe_{y} nanowires is applied according to Example 1.

FIG. 22 is a graph representing the changes in temperature of a catalyst plotted with respect to an increase in flow rate of hydrogen under the condition in which 1 vol % hydrogen flows in the thermochemical gas sensor to which the single thermoelectric device composed of the BiₓTe_{y} nanowires is applied according to Example 1, and FIG. 23 is a graph representing the changes in electromotive force occurring in the thermoelectric device plotted with respect to the increase in flow rate of hydrogen under the condition in which 1 vol % hydrogen flows in the thermochemical gas sensor to which the single thermoelectric device composed of the BiₓTe_{y} nanowires is applied according to Example 1.

FIG. 24 is a graph representing the changes in temperature of a catalyst plotted with respect to hydrogen concentrations, when hydrogen sensing takes place in a thermochemical gas sensor to which a thermoelectric device composed of P(SbₓTe_{y})—N(BiₓTe_{y}) junction nanowires is applied according to Example 2, and FIG. 25 is a graph representing the changes in electromotive force occurring in the thermoelectric device plotted with respect to hydrogen concentrations, when the hydrogen sensing takes place in the thermochemical gas sensor to which the thermoelectric device composed of the P(SbₓTe_{y})—N(BiₓTe_{y}) junction nanowires is applied according to Example 2.

FIG. 26 is a graph representing the changes in temperature of a catalyst plotted with respect to an increase in flow rate of hydrogen under the condition in which 1 vol % hydrogen flows, when the hydrogen sensing takes place in the thermochemical gas sensor to which the thermoelectric device composed of the P(SbₓTe_{y})—N(BiₓTe_{y}) junction nanowires is applied according to Example 2, and FIG. 27 is a graph representing the changes in electromotive force occurring in the thermoelectric device plotted with respect to the increase in flow rate of hydrogen under the condition in which 1 vol % hydrogen flows, when the hydrogen sensing takes place in the thermochemical gas sensor to which the thermoelectric device composed of the P(SbₓTe_{y})—N(BiₓTe_{y}) junction nanowires is applied according to Example 2.

FIG. 28 is a graph representing the changes in temperature at a low concentration when the hydrogen sensing takes place in the thermochemical gas sensor to which the thermoelectric device composed of the P(SbₓTe_{y})—N(BiₓTe_{y}) junction nanowires is applied according to Example 2, and FIG. 29 is a graph representing the changes in electromotive force at a low concentration when the hydrogen sensing takes place in the thermochemical gas sensor to which the thermoelectric device composed of the P(SbₓTe_{y})—N(BiₓTe_{y}) junction nanowires is applied according to Example 2.

### MODES OF THE INVENTION

A thermochemical gas sensor according to an exemplary embodiment of the present invention includes: a porous alumina template having top, bottom and side surfaces and including a plurality of pores penetrating the top and bottom surfaces; a seed layer with electric conductivity, which is formed on the bottom surface of the porous alumina template to cover the plurality of pores; a plurality of chalcogenide-based nanowires, which is in contact with the seed layer exposed through the plurality of pores and formed in the plurality of pores; an electrode, which is in contact with the chalcogenide-based nanowires and formed on the top surface of the porous alumina template; electrode wires electrically connected with the electrode; and a porous platinum-alumina composite or porous palladium-alumina composite, which is formed above the electrode and causes an exothermic reaction when in contact with a gas to be sensed wherein the chalcogenide-based nanowires consist of BiₓTe_{y} (1.5≦x≦2.5, 2.4≦y≦3.6), SbₓTe_{y} (1.5≦x≦2.5, 2.4≦y≦3.6) or (Bi₁₋ₓSbₓ)Te₃ (0<x<1).

A thermochemical gas sensor according to another exemplary embodiment of the present invention includes: a porous alumina template having top, bottom and side surfaces and including a plurality of pores penetrating the top and bottom surfaces; a seed layer with electric conductivity, which is formed on the bottom surface of the porous alumina template to cover the plurality of pores; a plurality of P-type chalcogenide-based nanowires, which is in contact with the seed layer exposed through the plurality of pores and formed in the plurality of pores; a plurality of N-type chalcogenide-based nanowires, which is in contact with the seed layer exposed through the plurality of pores and formed in the plurality of pores; an electrode, which is in contact with the P-type chalcogenide-based nanowires and the N-type chalcogenide-based nanowires and formed on the top surface of the porous alumina template; electrode wires electrically connected with the electrode; and a porous platinum-alumina composite or porous palladium-alumina composite, which is formed above the electrode and causes an exothermic reaction when in contact with a gas to be sensed, wherein the P-type chalcogenide-based nanowires consist of SbₓTe_{y} (1.5≦x≦2.5, 2.4≦y≦3.6) or (Bi₁₋ₓSbₓ)Te₃ (0<x<1), and the N-type chalcogenide-based nanowires consist of BiₓTe_{y} (1.5≦x≦2.5, 2.4≦y≦3.6).

A method of manufacturing a thermochemical gas sensor according to an exemplary embodiment of the present invention includes: preparing a porous alumina template having top, bottom and side surfaces and including a plurality of pores penetrating the top and bottom surfaces, and forming a seed layer with electric conductivity on the bottom surface of the porous alumina template to cover the plurality of pores; growing and forming a plurality of chalcogenide-based nanowires on the seed layer exposed through the plurality of pores using electrodeposition; forming an electrode in contact with the chalcogenide-based nanowires on the top surface of the porous alumina template; forming electrode wires electrically connected with the electrode; and forming a porous platinum-alumina composite or porous palladium-alumina composite above the electrode formed on the top surface of the porous alumina template, the composite causing an exothermic reaction when in contact with a gas to be sensed, wherein the chalcogenide-based nanowires consist of BiₓTe_{y} (1.5≦x≦2.5, 2.4≦y≦3.6), SbₓTe_{y} (1.5≦x≦2.5, 2.4≦y≦3.6) or (Bi₁₋ₓSbₓ)Te₃ (0<x<1), the electrodeposition uses an electrolyte containing at least one material selected from a bismuth (Bi) precursor and an antimony (Sb) precursor; a tellurium (Te) precursor; and an acid, and the acid is a material that can dissolve at least one selected from the bismuth (Bi) precursor and the antimony (Sb) precursor, and the tellurium (Te) precursor.

A method of manufacturing a thermochemical gas sensor according to another exemplary embodiment of the present invention includes: preparing a porous alumina template having top, bottom and side surfaces and including a plurality of pores penetrating the top and bottom surfaces, masking regions of the bottom surface of the porous alumina template, except the part in which chalcogenide-based nanowires are to be formed, and forming a seed layer with electric conductivity on the exposed part to cover a plurality of pores; covering a region in which N-type chalcogenide-based nanowires are to be formed on the top surface of the porous alumina template with a first mask, and growing and forming a plurality of P-type chalcogenide-based nanowires on the seed layer exposed through the plurality of pores using electrodeposition; covering the region in which the P-type chalcogenide-based nanowires have been formed on the top surface of the porous alumina template with a second mask, and growing and forming a plurality of N-type chalcogenide-based nanowires on the seed layer exposed through the plurality of pores by removal of the first mask using electrodeposition; forming an electrode in contact with the P-type chalcogenide-based nanowires and the N-type chalcogenide-based nanowires on the top surface of the porous alumina template; forming electrode wires electrically connected with the electrode; and forming a porous platinum-alumina composite or porous palladium-alumina composite above the electrode formed on the top surface of the porous alumina template, the composite causing an exothermic reaction when in contact with a gas to be sensed, wherein the P-type chalcogenide-based nanowires consist of SbₓTe_{y} (1.5≦x≦2.5, 2.4≦y≦3.6) or (Bi₁₋ₓSbₓ)Te₃ (0<x<1), the N-type chalcogenide-based nanowires consist of BiₓTe_{y} (1.5≦x≦2.5, 2.4≦y≦3.6), the electrodeposition for forming the P-type chalcogenide-based nanowires uses an electrolyte containing one or both of an antimony (Sb) precursor and a bismuth (Bi) precursor, a tellurium (Te) precursor and an acid, the electrodeposition for forming the N-type chalcogenide-based nanowires uses a bismuth (Bi) precursor, a tellurium (Te) precursor and an acid, and the acid is a material that can dissolve an antimony (Sb) precursor, a bismuth (Bi) precursor and a tellurium (Te) precursor.

### BEST MODE

Hereinafter, exemplary embodiments of the present invention will be described in detail with reference to the accompanying drawings. However, the following exemplary embodiments are provided for those of ordinary skill in the art to fully understand the present invention. Therefore, the exemplary embodiments can be modified in various forms, and the scope of the present invention is not limited to the exemplary embodiments that will be described below.

Hereinafter, the nano means the size of a nanometer (nm) unit, ranging from 1 to 1,000 nm, and the nanowire refers to a wire having a diameter ranging from 1 to 1,000 nm.

Pores are classified into three types depending on a pore diameter of a porous material, for example, a micropore having a diameter of 2 nm or less, a mesopore having a diameter of 2 to 50 nm, and a macropore having a diameter of 50 nm or more, as defined by International union of Pure and Applied Chemistry (IUPAC). As referred to later, the macropore refers to a pore having a pore diameter of 50 nm or more, and the mesopore refers to a pore having a pore diameter of 2 to 50 nm according to IUPAC.

The present invention provides a thermochemical gas sensor manufactured based on a thermoelectric device consisting of chalcogenide-based nanowires and a method of manufacturing the same.

The thermochemical gas sensor of the present invention is manufactured by forming a single thermoelectric device or a P-N junction thermoelectric device exhibiting maximized thermoelectric properties by selectively plating chalcogenide-based nanowires known in a porous anodic alumina template using electrodeposition, and binding a porous catalyst-alumina composite (a porous platinum-alumina composite or a porous palladium-alumina composite) causing an exothermic reaction when in contact with a gas to be sensed. The thermochemical gas sensor of the present invention is a new type of thermoelectric nanowire array-based thermochemical gas sensor that can sense a gas, and identify and evaluate a gas sensing property.

A thermochemical gas sensor according to a first exemplary embodiment of the present invention includes a porous alumina template having top, bottom and side surfaces and including a plurality of pores penetrating the top and bottom surfaces, a seed layer with electric conductivity, which is formed on the bottom surface of the porous alumina template to cover the plurality of pores, a plurality of chalcogenide-based nanowires, which is in contact with the seed layer exposed through the plurality of pores and formed in the plurality of pores, an electrode, which is in contact with the chalcogenide-based nanowires and formed on the top surface of the porous alumina template, electrode wires electrically connected with the electrode, and a porous platinum-alumina composite or porous palladium-alumina composite, which is formed above the electrode and causes an exothermic reaction when in contact with a gas to be sensed (e.g., a hydrogen gas), wherein the chalcogenide-based nanowires consist of BiₓTe_{y} (1.5≦x≦2.5, 2.4≦y≦3.6), SbₓTe_{y} (1.5≦x≦2.5, 2.4≦y≦3.6) or (Bi₁₋ₓSbₓ)Te₃ (0<x<1).

A thermochemical gas sensor according to a second exemplary embodiment of the present invention includes a porous alumina template having top, bottom and side surfaces and including a plurality of pores penetrating the top and bottom surfaces, a seed layer with electric conductivity, which is formed on the bottom surface of the porous alumina template to cover the plurality of pores, a plurality of P-type chalcogenide-based nanowires, which is in contact with the seed layer exposed through the plurality of pores and formed in the plurality of pores, a plurality of N-type chalcogenide-based nanowires, which is in contact with the seed layer exposed through the plurality of pores and formed in the plurality of pores, an electrode, which is in contact with the P-type chalcogenide-based nanowires and the N-type chalcogenide-based nanowires and formed on the top surface of the porous alumina template, electrode wires electrically connected with the electrode, and a porous platinum-alumina composite or porous palladium-alumina composite, which is formed above the electrode and causes an exothermic reaction when in contact with a gas to be sensed (e.g., a hydrogen gas), wherein the P-type chalcogenide-based nanowires consist of SbₓTe_{y} (1.5≦x≦2.5, 2.4≦y≦3.6) or (Bi₁₋ₓSbₓ)Te₃ (0<x<1), and the N-type chalcogenide-based nanowires consist of BiₓTe_{y} (1.5≦x≦2.5, 2.4≦y≦3.6).

The seed layer may have a thickness of 10 to 1000 nm, and consist of at least one metal selected from gold (Au), silver (Ag) and copper (Cu).

The pores may have an average diameter of 10 to 1000 nm, and the chalcogenide-based nanowires may have an average diameter of 1 to 500 nm, which is smaller than the average diameter of the pores.

The length of the chalcogenide-based nanowires may be the same as or smaller than the depth of the pores.

The porous platinum-alumina composite or porous palladium-alumina composite may be a porous material having a plurality of macropores and a plurality of mesopores.

In the porous platinum-alumina composite or porous palladium-alumina composite, alumina may be γ-alumina.

The porous platinum-alumina composite may be a material containing 0.1 to 12 vol % of platinum (Pt) and 88 to 99.9 vol % of alumina with consideration for the exothermic reaction with a gas to be sensed, and the porous palladium-alumina composite may be a material containing 0.1 to 12 vol % of palladium (Pd) and 88 to 99.9 vol % of alumina with consideration for the exothermic reaction with a gas to be sensed.

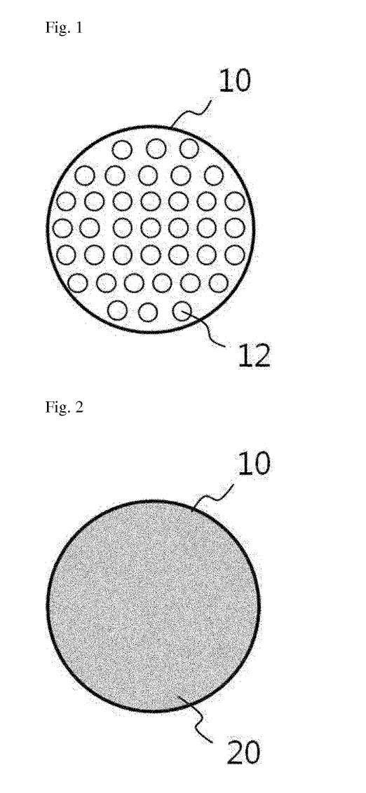

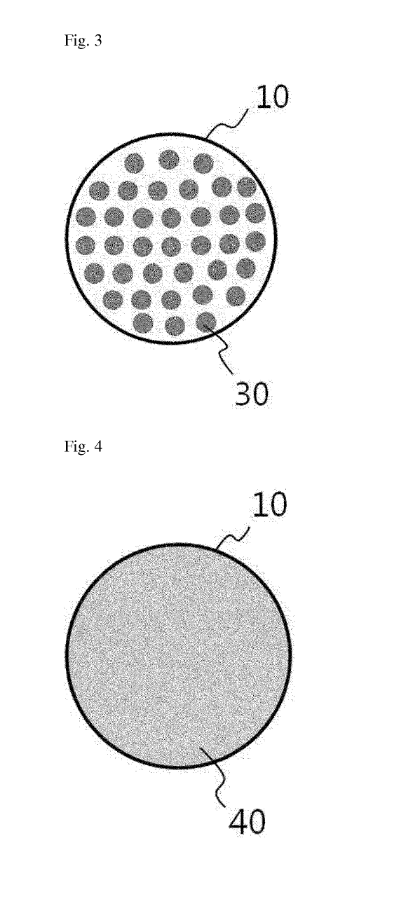

Hereinafter, a method of manufacturing a thermochemical gas sensor according to a first exemplary embodiment of the present invention will be described in detail. FIGS. 1 to 4 are schematic diagrams illustrating a process of manufacturing a thermochemical gas sensor using a single thermoelectric device according to a first exemplary embodiment of the present invention.

Referring to FIGS. 1 to 4, a porous alumina template 10 having top, bottom and side surfaces and including a plurality of pores 12 penetrating the top and bottom surfaces is prepared. The pores 12 may have an average diameter of 10 to 1000 nm.

A seed layer 20 with electric conductivity is formed on the bottom surface of the porous alumina template 10 to cover the plurality of pores. The seed layer 20 may have a thickness of 10 to 1000 nm, and consist of at least one metal selected from gold (Au), silver (Ag) and copper (Cu). The seed layer 20 may be formed by deposition in various methods, for example, sputtering. The seed layer 20 is formed to cover the pores 12 in the bottom surface of the porous alumina template 10.

A plurality of chalcogenide-based nanowires 30 are grown on the seed layer 20 exposed through the plurality of pores 12 on the top surface of the porous alumina template using electrodeposition.

The chalcogenide-based nanowires 30 may consist of BiₓTe_{y} (1.5≦x≦2.5, 2.4≦y≦3.6), SbₓTe_{y} (1.5≦x≦2.5, 2.4≦y≦3.6) or (Bi₁₋ₓSbₓ)Te₃ (0<x<1).

In the present invention, the chalcogenide-based nanowires 30 are formed in the porous alumina template 10 using electrodeposition that facilitates the synthesis of a nano structure at a low cost. The electrodeposition is a method of easily synthesizing a desired type of the chalcogenide-based nanowires 30 having a desired composition and uniform lengths at a low process cost, and thus the sensor can be downsized on a nano scale, and the thermoelectric material-based hydrogen gas sensor has a wide concentration range in which hydrogen can be sensed, and is not accompanied with physical/chemical changes such as a phase change in a thermoelectric material even when the sensor is repeatedly exposed to a hydrogen gas. In addition, by adjusting the pores 12 of the porous alumina template 10 and the plating conditions, the chalcogenide-based nanowires 30 having desired diameter, length and composition may be synthesized.

The electrodeposition uses an electrolyte containing at least one material selected from a bismuth (Bi) precursor and an antimony (Sb) precursor; a tellurium (Te) precursor; and an acid, and the acid is a material that can dissolve at least one material selected from the bismuth (Bi) precursor and the antimony (Sb) precursor; and the tellurium (Te) precursor. The electrodeposition may be performed by applying a voltage to a two- or three-electrode system using a rectifier.

The bismuth (Bi) precursor may be Bi(NO₃)₃.5H₂O, the antimony (Sb) precursor may be Sb₂O₃, the tellurium (Te) precursor may be TeO₂, and the acid may be HNO₃.

When the chalcogenide-based nanowires 30 consist of SbₓTe_{y} (1.5≦x≦2.5, 2.4≦y≦3.6) or (Bi₁₋ₓSbₓ)Te₃ (0<x<1), prior to the formation of the electrode 40 after the growth of the chalcogenide-based nanowires 30, annealing may be performed on the chalcogenide-based nanowires 30 at 100 to 300° C.

The chalcogenide-based nanowires 30 may be formed to have an average diameter of 1 to 500 nm, which is smaller than that of the pores 12, and the length of the chalcogenide-based nanowires 30 may be formed the same as or smaller than the depth of the pores 12.

The electrode 40 in contact with the chalcogenide-based nanowires 30 is formed on the top surface of the porous alumina template 10. The electrode 40 is formed by electroplating at least one metal selected from gold (Au), silver (Ag) and copper (Cu), and the electroplating may be performed by applying a current to a two-electrode system using a rectifier, while stirring with a magnetic bar.

The electrode wires electrically connected with the electrode 40 are formed. The electrode wires may also be electrically connected to the seed layer in order to evaluate the properties of the thermoelectric device. The electrode wires may be formed with a copper wire, for example, using a silver paste.

The porous platinum-alumina composite or porous palladium-alumina composite causing an exothermic reaction when in contact with a gas to be sensed (e.g., a hydrogen gas) is formed on the electrode 40 formed on the top surface of the porous alumina template 10. In the porous platinum-alumina composite or porous palladium-alumina composite, alumina may be γ-alumina. The porous platinum-alumina composite may be a material containing 0.1 to 12 vol % of platinum (Pt) and 88 to 99.9 vol % of alumina with consideration for the exothermic reaction with the gas to be sensed, and the porous palladium-alumina composite may be a material containing 0.1 to 12 vol % of palladium (Pd) and 88 to 99.9 vol % of alumina with consideration for the exothermic reaction with the gas to be sensed.

Hereinafter, a method of preparing the porous platinum-alumina composite or porous palladium-alumina composite will be described.

A mixed solution of styrene and distilled water is prepared, a polystyrene solution is synthesized by adding potassium persulfate to the mixed solution, and the polystyrene solution is dried, thereby obtaining colloidal crystals. A precursor solution of the platinum-alumina composite or palladium-alumina composite is synthesized, the colloidal crystals obtained by drying are immersed in the precursor solution of the platinum-alumina composite or palladium-alumina composite, and the colloidal crystals immersed in the precursor solution of the platinum-alumina composite or palladium-alumina composite are dried and calcined to remove the polystyrene colloidal crystals.

The precursor solution of the platinum-alumina composite may be a solution containing aluminumisopropoxide (C₉H₂₁O₃Al) and chloroplatinic acid (H₂PtCl₆), and the precursor solution of the palladium-alumina composite may be a solution containing aluminumisopropoxide (C₉H₂₁O₃Al) and chloropalladic acid (H₂PdCl₆).

The porous platinum-alumina composite or porous palladium-alumina composite prepared as described above is a porous material having a plurality of macropores and a plurality of mesopores, and causes the exothermic reaction in contact with the gas to be sensed (e.g., a hydrogen gas).

According to the above-described method of preparing the porous platinum-alumina composite or porous palladium-alumina composite, macropores having regular arrangement may be formed by removing the polystyrene colloidal crystals used as a template. A platinum-alumina composite or palladium-alumina composite having macro-mesopores in which mesopores unique to alumina as well as such macropores are formed to work together may be synthesized. As the macro-mesopores are formed in the platinum-alumina composite or palladium-alumina composite, a diffusion rate of molecules is increased, thereby achieving a fast response characteristic and high sensitivity.

Polystyrene is present in the form of beads in the polystyrene solution, and the size of the beads is relevant to reaction time. The size of the macropores is relevant to the size of the colloidal crystals, and thus the size of the beads, and therefore, as the size of the beads is adjusted by adjusting the reaction time, the amount of the potassium persulfate, and a ratio of the distilled water to the styrene, the size of the macropores can be controlled.

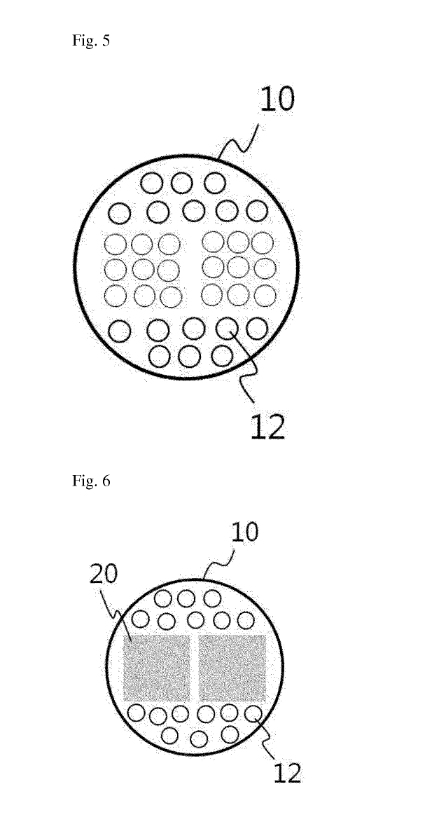

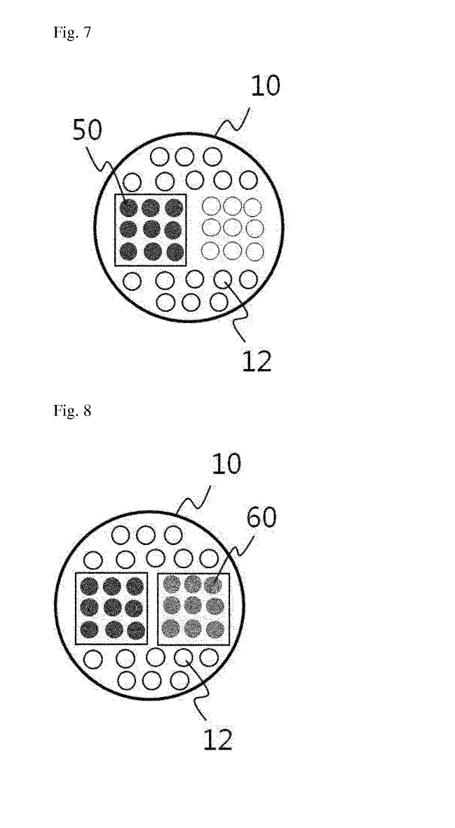

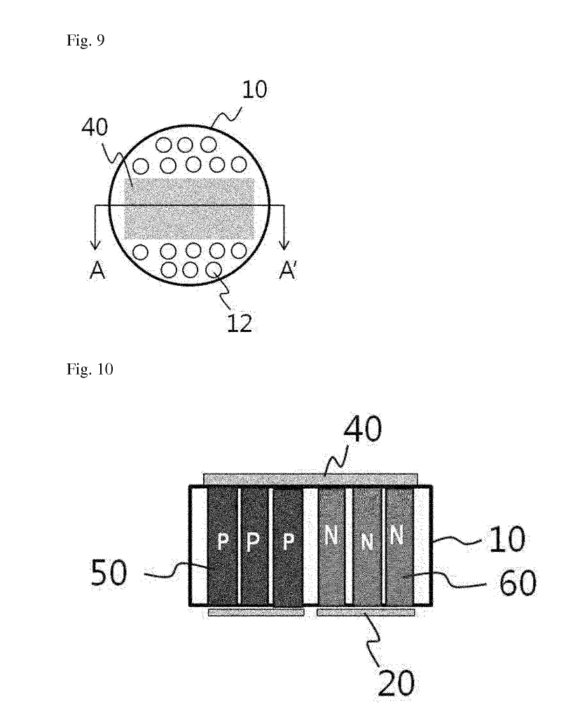

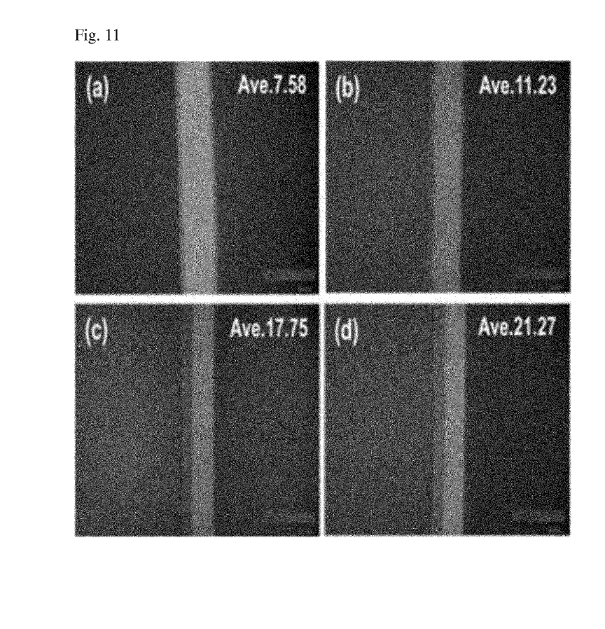

Hereinafter, a method of manufacturing a thermochemical gas sensor according to a second exemplary embodiment of the present invention will be described in detail. FIGS. 5 to 10 are schematic drawings illustrating a process of manufacturing a thermochemical gas sensor using a P-N junction thermoelectric device according to a second exemplary embodiment of the present invention. FIG. 10 is a cross-sectional view taken along line A-A′ of FIG. 9.

Referring to FIGS. 5 to 10, a porous alumina template 10 having top, bottom and side surfaces and including a plurality of pores 12 penetrating the top and bottom surfaces is prepared. The pores 12 may have an average diameter of 10 to 1000 nm.

Following masking of regions of the bottom surface of the porous alumina template 10, excluding the part in which chalcogenide-based nanowires are to be formed, a seed layer 20 with electric conductivity is formed on an exposed part to cover the plurality of pores. The seed layer 20 may have a thickness of 10 to 1000 nm, and consist of at least one metal selected from gold (Au), silver (Ag) and copper (Cu). The seed layer 20 may be formed by deposition in various methods, for example, sputtering. The seed layer 20 is formed to cover the pores 12 in the bottom surface of the porous alumina template 10.

A region of the top surface of the porous alumina template 10 in which N-type chalcogenide-based nanowires 60 are to be formed is covered with a first mask, and a plurality of P-type chalcogenide-based nanowires 50 is grown on the seed layer 20 exposed through the plurality of pores 12 on the top surface of the porous alumina template using electrodeposition.

The region in which the P-type chalcogenide-based nanowires 50 are formed is covered with a second mask, and a plurality of N-type chalcogenide-based nanowires 60 is grown on the seed layer 20 exposed through the plurality of pores 12 by removing the first mask using electrodeposition.

The P-type chalcogenide-based nanowires 50 may consist of SbₓTe_{y} (1.5≦x≦2.5, 2.4≦y≦3.6) or (Bi₁₋ₓSbₓ)Te₃ (0<x<1), and the N-type chalcogenide-based nanowires 60 may consist of BiₓTe_{y} (1.5≦x≦2.5, 2.4≦y≦3.6).

In the present invention, the chalcogenide-based nanowires are formed in the porous alumina template 10 using electrodeposition that facilitates the synthesis of a nano structure at a low cost. The electrodeposition is a method of easily synthesizing a desired type of the chalcogenide-based nanowires having a desired composition and uniform lengths at a low process cost, and thus the sensor can be downsized on a nano scale, and the thermoelectric material-based hydrogen gas sensor has a wide concentration range in which hydrogen can be sensed, and is not accompanied with physical/chemical changes such as a phase change in a thermoelectric material even when the sensor is repeatedly exposed to a hydrogen gas. In addition, the chalcogenide-based nanowires having desired diameter, length and composition may be synthesized by adjusting the pores 12 of the porous alumina template 10 and the plating conditions.

The electrodeposition for forming the P-type chalcogenide-based nanowires 50 uses an electrolyte containing one or both of an antimony (Sb) precursor and a bismuth (Bi) precursor, a tellurium (Te) precursor and an acid, the electrodeposition for forming the N-type chalcogenide-based nanowires 60 uses an electrolyte containing a bismuth (Bi) precursor, a tellurium (Te) precursor and an acid, and the acid is a material that can dissolve an antimony (Sb) precursor, a bismuth (Bi) precursor and a tellurium (Te) precursor. The electrodeposition may be performed by applying a voltage to a two- or three-electrode system using a rectifier.

The bismuth (Bi) precursor may be Bi(NO₃)₃.5H₂O, the antimony (Sb) precursor may be Sb₂O₃, the tellurium (Te) precursor may be TeO₂, and the acid may be HNO₃.

When the chalcogenide-based nanowires consist of SbₓTe_{y} (1.5≦x≦2.5, 2.4≦y≦3.6) or (Bi₁₋ₓSbₓ)Te₃ (0<x<1), prior to the formation of the electrode 40 after the growth of the chalcogenide-based nanowires annealing may be performed on the chalcogenide-based nanowires at 100 to 300° C.

The chalcogenide-based nanowires may be formed to have an average diameter of 1 to 500 nm smaller than that of the pores 12, and the length of the chalcogenide-based nanowires may be formed the same as or smaller than the depth of the pores 12.

An electrode 40 in contact with the P-type chalcogenide-based nanowires 50 and the N-type chalcogenide-based nanowires 60 is formed on the top surface of the porous alumina template 10. The electrode 40 may be formed by electroplating at least one metal selected from gold (Au), silver (Ag) and copper (Cu), and the electroplating may be performed by applying a current to a two-electrode system using a rectifier, while stirring with a magnetic bar.

Electrode wires electrically connected with the electrode 40 are formed. The electrode wires may also be electrically connected to the seed layer to evaluate the properties of the thermoelectric device. The electrode wires may be formed with copper wires, for example, using a silver paste.

A porous platinum-alumina composite or porous palladium-alumina composite causing an exothermic reaction when in contact with a gas to be sensed (e.g., a hydrogen gas) is formed above the electrode 40 formed on the top surface of the porous alumina template 10. In the porous platinum-alumina composite or porous palladium-alumina composite, alumina may be γ-alumina. The porous platinum-alumina composite may be a material containing 0.1 to 12 vol % of platinum (Pt) and 88 to 99.9 vol % of alumina with consideration for an exothermic reaction with a gas to be sensed, and the porous palladium-alumina composite may be a material containing 0.1 to 12 vol % of palladium (Pd) and 88 to 99.9 vol % of alumina with consideration for an exothermic reaction with a gas to be sensed. The porous platinum-alumina composite or porous palladium-alumina composite may be formed by the same method as described above, and hence descriptions of the method will be omitted.

The thermochemical gas sensor using chalcogenide-based nanowires of the present invention uses the principle in which an electromotive force is generated by the change in temperature, and by oxidation of hydrogen with the porous catalyst-alumina composite (a porous platinum-alumina composite or a porous palladium-alumina composite) and the exothermic reaction, water is generated as a by-product and heat is generated in the porous catalyst-alumina composite, and then the heat is transferred to the chalcogenide-based nanowires, which are thermoelectric materials, generating an electromotive force.

The BiₓTe_{y} (1.5≦x≦2.5, 2.4≦y≦3.6), SbₓTe_{y} (1.5≦x≦2.5, 2.4≦y≦3.6) or (Bi₁₋ₓSbₓ)Te₃ (0<x<1) for forming the chalcogenide-based nanowires are a material exhibiting a high thermoelectric property at room temperature, and may be easily synthesized using electrodeposition. The thermoelectric materials exhibiting a thermoelectric property in a temperature range suitable for an operation temperature may be easily synthesized by the electrodeposition.

In addition, various types of desired gases can be sensed depending on changes in the porous platinum-alumina composite or porous palladium-alumina composite in response to a gas to be sensed (e.g., a hydrogen gas). Also, since a temperature changed in sensing of a gas and a subtle change in electromotive force can be examined, gas sensing can also be utilized as a method of evaluating a thermoelectric figure of merit using a gas.

Since the method of manufacturing a thermochemical gas sensor according to the present invention uses electrodeposition employing a low-priced synthesis method, a sensor is manufactured at room temperature without using a high vacuum and high temperature process having a high process cost, and therefore the amount of a material applied to each device can be minimized, thereby ensuring price competitiveness.

Also, with the development and increase in demand for hydrogen fuel cells getting the spotlight as future clean energy, it is considered that stability to the fuel cells can be ensured and an energy source can be produced from a thermoelectric material using waste heat in the automotive field.

Also, since a hydrogen battery is also used in the field of aerospace technology, for example, a satellite, a space shuttle, etc., the development of a suitable hydrogen sensor for the above purpose is needed, and it is necessary to study methods of mass-producing a compact and high-sensitive hydrogen sensor in accordance with micro electro mechanical systems (MEMS), which is one of the microscopic circuit manufacturing technology. Moreover, it is considered that the gas sensor of the present invention can be applied to MEMS technology through the downsizing of the thermochemical gas sensor manufactured according to the present invention and the development of integrated application/coating technology of a catalyst using inkjet printing.

Hereinafter, examples according to the present invention will be described in detail, and the present invention is not limited to the examples that will be described below.

### Example 1

To manufacture a thermochemical gas sensor in this example, a porous alumina template having a diameter of 12 mm and a pore diameter of 200 nm was used as a matrix of the sensor, and electrodeposition was used to form chalcogenide-based nanowires in the porous alumina template.

To form a single thermoelectric device in the porous alumina template, a sputtering process was performed on a bottom surface of the alumina template, thereby forming a gold seed layer. The height of the gold seed layer formed as described above was detected at approximately 200 nm.

The gold seed layer exposed through pores formed in the top surface of the porous alumina template was grown by electroplating for 8 hours with a voltage of 75 mV in a three-electrode system using a predetermined rectifier to form BiₓTe_{y} (1.5≦x≦2.5, 2.4≦y≦3.6) nanowires. Here, as an electrolyte, a mixture of 1 M of HNO₃, 70 mM of Bi(NO₃)₃ 5H₂O and 10 mM of TeO₂ was used.

An electrode in contact with the BiₓTe_{y} nanowires was formed. The electrode was formed by electroplating a gold layer. The electroplating for forming the electrode was performed by applying a current of 1 mA in a two-electrode system using a predetermined rectifier, while stirring at 250 rpm with a magnetic bar.

Prior to hydrogen sensing, for connection of a nanovoltmeter measuring an electromotive force generated in a thermoelectric device, copper wires were connected to the electrode and the seed layer using a silver paste.

A porous platinum-alumina composite was formed above the electrode on which the copper wires had been formed. The porous platinum-alumina composite was a catalyst consisting of 2 vol % of platinum (Pt) and 98 vol % of γ-alumina, and directly applied to a top surface of the electrode at 0.05 g. For uniform heat transfer, the porous platinum-alumina composite was uniformly spread on the resulting product in which the electrode had been formed.

The porous platinum-alumina composite was manufactured by the method that will be described below.

First, polystyrene beads for forming macropores were manufactured. 10 ml of styrene was washed with 10 ml of 0.1M sodium hydroxide (NaOH) aqueous solution five times, and then washed again with 10 ml of distilled water five times. At the same time, 100 ml of distilled water was added to a three-neck flask, and heated at 70° C. under a nitrogen atmosphere. Subsequently, 10 ml of previously washed styrene was added to distilled water at 70° C. and stirred. Subsequently, 0.04 g of potassium persulfate was added to a mixed solution of styrene and distilled water and stirred for 28 hours under a nitrogen atmosphere at 70° C., thereby synthesizing a solution in which polystyrene was present in the form of beads.

2.0425 g of aluminumisopropoxide (C₉H₂₁O₃Al) was added to 18 ml of distilled water at 80° C., and stirred for one hour. Here, 10 wt % of nitric acid (HNO₃) was added to maintain the pH of the mixture to 5.5, and stirred at 90° C. for 5 hours. After reducing a temperature, 1.303 ml of chloroplatinic acid (H₂PtCl₆) was added, and then stirred for one hour, thereby synthesizing a precursor solution for a platinum-alumina composite.

The synthesized polystyrene solution was centrifuged at 4000 rpm for three hours and dried, thereby obtaining colloidal crystals. The colloidal crystals obtained as such were immersed in the previously synthesized precursor solution of the platinum-alumina composite for one hour. Afterward, the colloidal crystals were taken from the precursor solution of the platinum-alumina composite, and the excessive precursor remaining on the periphery was wiped out and then dried at 100° C. for 12 hours. After drying, a template material, which is polystyrene colloidal crystals, was removed by calcining at 600° C. for 6 hours, thereby forming a porous platinum-alumina composite.

### Example 2

A porous alumina template having a diameter of 12 mm and a pore diameter of 200 nm was used as a matrix of the sensor to manufacture a thermochemical gas sensor in this example, and electrodeposition was used to form chalcogenide-based nanowires in the porous alumina template.

A process of forming a P-N junction thermoelectric device in the porous alumina template was performed.

First, masking was performed using stencil, except the part in which the nanowires were to be plated, and a sputtering process was performed on the exposed part, thereby forming a gold seed layer. The height of the gold seed layer formed as such was detected at approximately 200 nm.

Afterward, to synthesize P-type SbₓTe_{y} (1.5≦x≦2.5, 2.4≦y≦3.6) nanowires, the part in which N-type BiₓTe_{y} (1.5≦x≦2.5, 2.4≦y≦3.6) nanowires were to be synthesized was masked using a microstop, and the SbₓTe_{y} nanowires were grown and formed on the gold seed layer exposed through pores on the top surface of the porous alumina template by plating for 5 hours with applying a voltage of 0.17 V in a three-electrode system using a predetermined rectifier. Here, as an electrolyte, a mixture of 1M HNO₃, 5 mM Sb₂O₃, and 10 mM TeO₂, 0.5M C₄H₆O₆ was used.

In order to synthesize BiₓTe_{y} nanowires, masking was performed on the part in which the SbₓTe_{y} nanowires had been synthesized using a microstop, and the BiₓTe_{y} nanowires was grown and formed on the gold seed layer exposed through the pores on the top surface of the porous alumina template by applying a voltage of 75 mV in the three-electrode system using a predetermined rectifier for 8 hours under stirring at 120 rpm. Here, as an electrolyte, a mixture of 1M HNO₃, 70 mM Bi(NO₃)₃.5H₂O and 10 mM TeO₂ was used.

An electrode in contact with the SbₓTe_{y} nanowires and the BiₓTe_{y} nanowires was formed. The electrode was formed by electroplating a gold layer. The electroplating for forming the electrode was performed by applying a current of 1 mA in a two-electrode system using a predetermined rectifier, while stirring at 250 rpm with a magnetic bar.

Prior to hydrogen sensing, for connection of a nanovoltmeter measuring an electromotive force generated in a thermoelectric device, copper wires were connected to the electrode and the seed layer using a silver paste.

A porous platinum-alumina composite was formed on the electrode in which the copper wires had been formed. The porous platinum-alumina composite was a catalyst consisting of 2 vol % of platinum (Pt) and 98 vol % of γ-alumina, and 0.05 g of the porous platinum-alumina composite was directly applied to the top surface of the electrode. For uniform heat transfer, the porous platinum-alumina composite was uniformly spread on the resulting product to which the electrode had been applied.

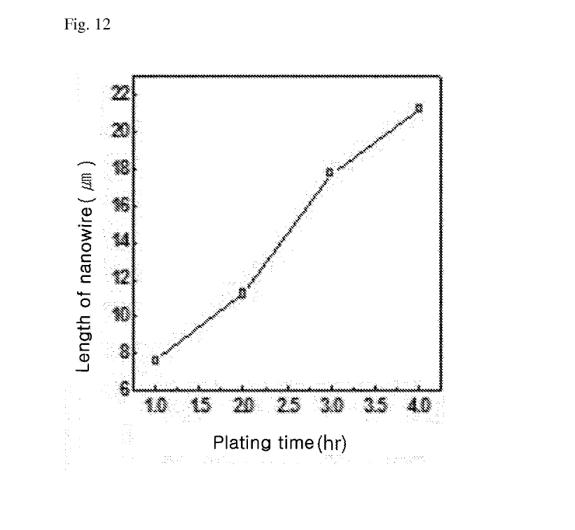

FIG. 11 is an optical microscope image of a cross-section of a porous alumina template that is transversely cut after BiₓTe_{y} nanowires are formed in the porous alumina template using electrodeposition according to Example 1, and FIG. 12 is a graph representing the lengths of the BiₓTe_{y} nanowires as a function of plating time, wherein the BiₓTe_{y} nanowires are synthesized in the porous alumina template through electroplating according to Example 1.

Referring to FIGS. 11 and 12, it was confirmed that the BiₓTe_{y} nanowires were grown by a length of approximately 5.31 μm per hour on average.

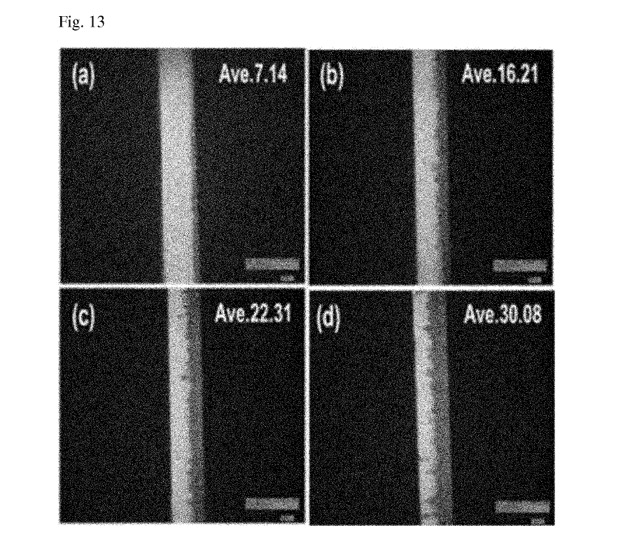

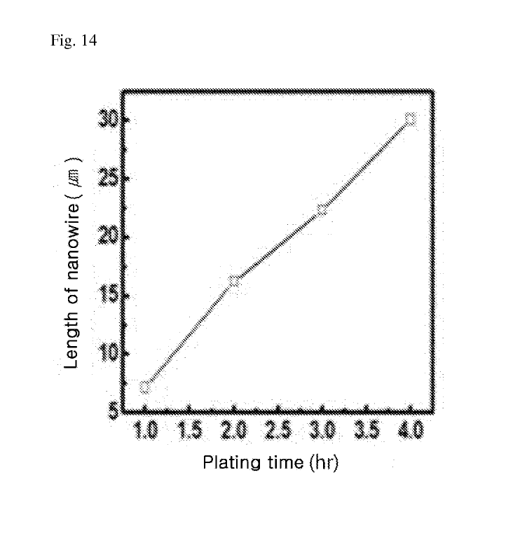

FIG. 13 is an optical microscope image of a cross-section of a porous alumina template that is transversely cut after SbₓTe_{y} nanowires are synthesized in the porous alumina template through electroplating according to Example 2, and FIG. 14 is a graph representing the lengths of the SbₓTe_{y} nanowires as a function of plating time, wherein the SbₓTe_{y} nanowires are synthesized in the porous alumina template through electroplating according to Example 2.

Referring to FIGS. 13 and 14, it was confirmed that the SbₓTe_{y} nanowires were grown by a length of approximately 7.52 μm per hour on average.

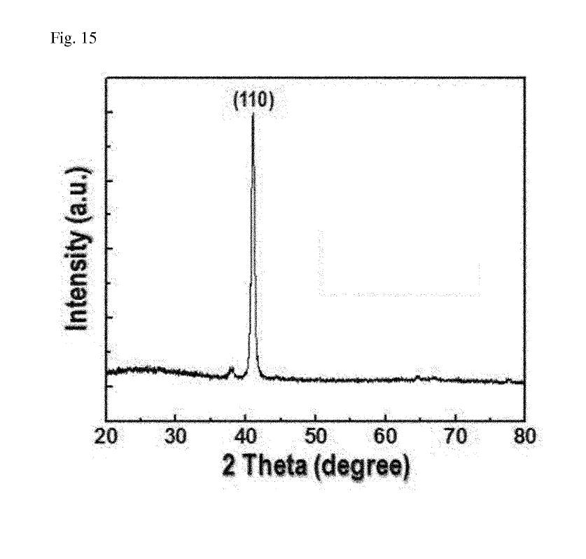

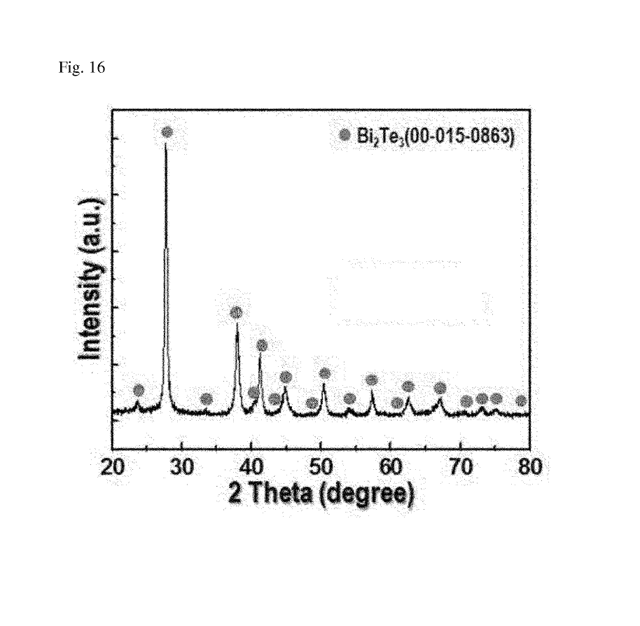

To examine a phase of the synthesized nanowires, an XRD pattern was produced. FIGS. 15 and 16 are graphs representing the XRD results of the BiₓTe_{y} nanowires synthesized by electroplating according to Example 1.

Referring to FIGS. 15 and 16, when an XRD pattern was produced without removal of a porous alumina template (refer to FIG. 15), it was confirmed that the BiₓTe_{y} nanowires were grown to have preferred orientation in (110) direction, and when an XRD patter was measured only with the BiₓTe_{y} nanowires obtained by removing a porous alumina template using 1M NaOH (refer to FIG. 16), it was confirmed that the BiₓTe_{y} nanowires had the phase of Bi₂Te₃ (JCPDS 00-015-0863).

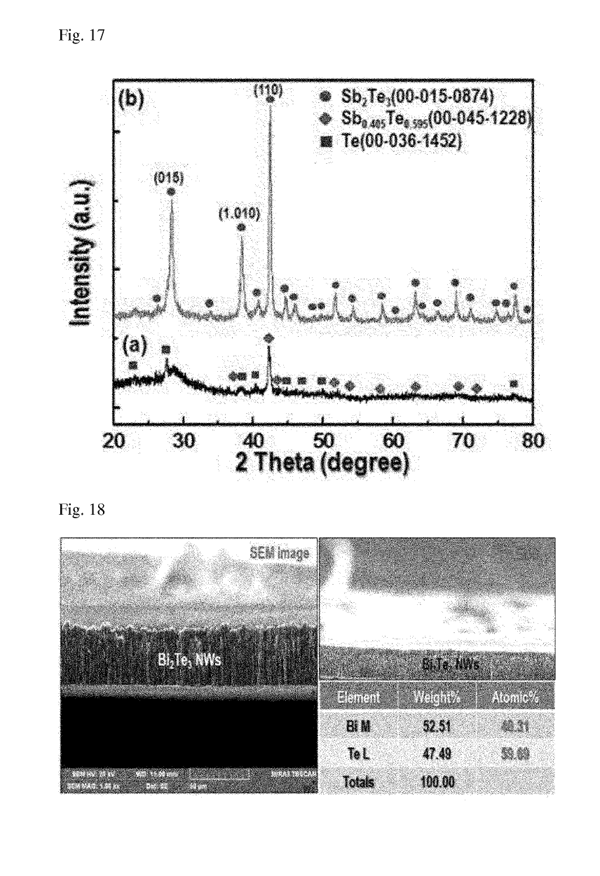

FIG. 17 is a graph representing the XRD results of the SbₓTe_{y} nanowires synthesized by electroplating according to Example 2.

Referring to FIG. 17, from the XRD analysis results for the SbₓTe_{y} nanowires after plating, the phase in which Sb_{0.405}Te_{0.595} and tellurium were mixed was examined.

Accordingly, to form the Sb₂Te₃ phase, after the XRD analysis of FIG. 17 was conducted, an annealing process for the SbₓTe_{y} nanowires was performed. When XRD analysis was conducted after the annealing in an atmospheric ambient at 120° C. for one hour, it was confirmed that the SbₓTe_{y} nanowires had the Sb₂Te₃ (JCPDS 00-015-0874) phase.

To confirm the shape and composition of the nanowires, a field emission-scanning electron microscope (FE-SEM) and energy dispersive spectroscopy (EDS) analyses were performed.

FIG. 18 shows the FE-SEM images and EDS analysis result for the BiₓTe_{y} nanowires synthesized using electroplating according to Example 1.

Referring to FIG. 18, according to an EDS analysis result, it can be confirmed that the result almost corresponded to the composition of Bi₂Te₃. It corresponds to the XRD data of FIGS. 15 and 16.

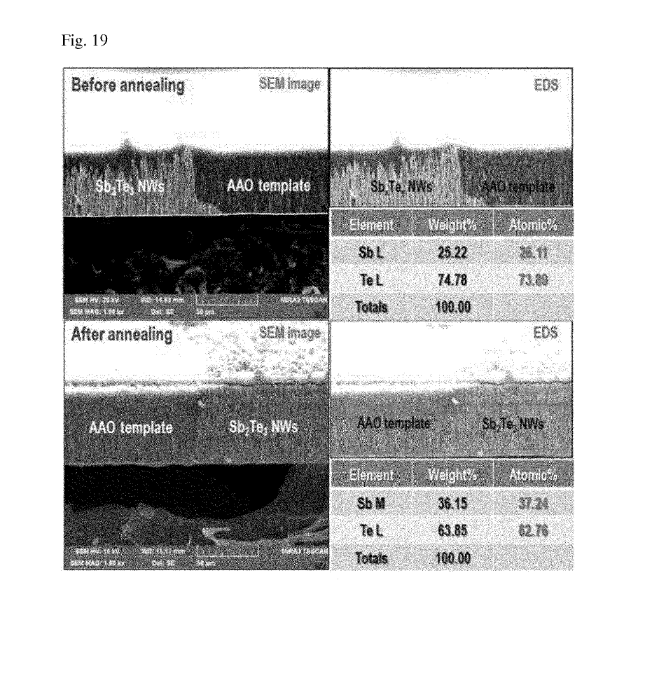

FIG. 19 shows the FE-SEM images and EDS analysis result, obtained before and after the annealing, for the SbₓTe_{y} nanowires synthesized by electroplating according to Example 2. The annealing was performed in an atmospheric ambience at 120° C. for one hour after the XRD of the SbₓTe_{y} nanowires shown in FIG. 17 was observed and FE-SEM observation and EDS analysis were performed. In FIG. 19, the “AAO template” stands for a porous alumina template, and the “Sb₂Te₃ NWs” stands for Sb₂Te₃ nanowires.

Referring to FIG. 19, before the annealing, an atomic ratio was approximately 26.11:73.89, which was much different from the composition of Sb₂Te₃. However, after the annealing was performed in an atmospheric ambience at 120° C. for one hour, the atomic ratio was 37.34:62.76, which was close to the composition of Sb₂Te₃. This corresponds to the XRD data of FIG. 17.

Properties of sensing hydrogen by the thermochemical gas sensors manufactured according to Examples 1 and 2 were evaluated. For sensing, a cycle including the flow of a hydrogen gas for 180 seconds and the cut-off of the hydrogen gas for 600 seconds was repeated. A small time difference between the temperature graph and the electromotive force graph was made by measuring an electromotive force after warm-up in argon and oxygen atmospheres for approximately 3 minutes in order to stabilize the atmospheres during the measurement of a temperature.

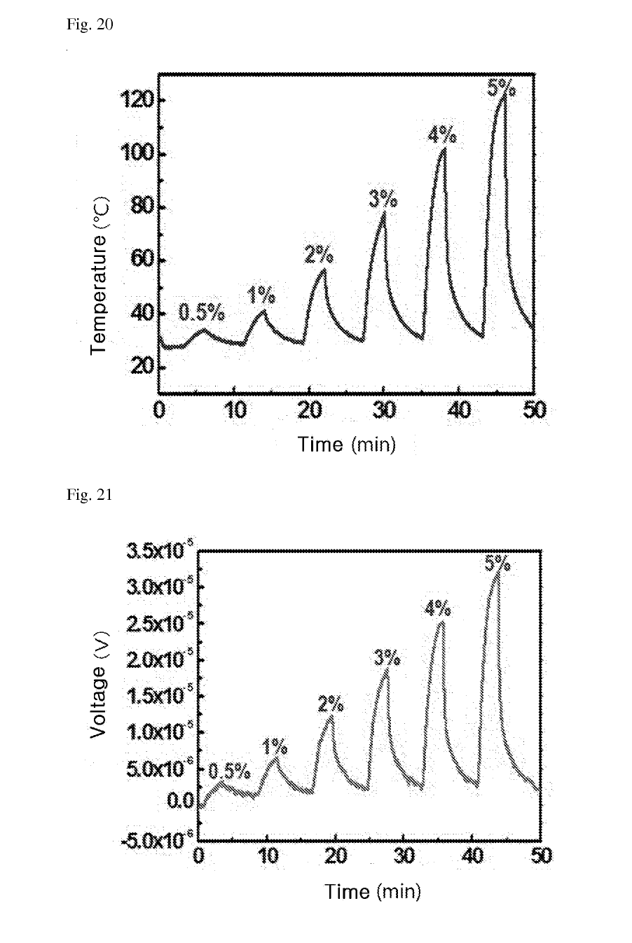

FIG. 20 is a graph representing the changes in temperature of the porous platinum-alumina composite plotted with respect to hydrogen concentrations when hydrogen sensing takes place in a thermochemical gas sensor to which a single thermoelectric device composed of the BiₓTe_{y} nanowires is applied according to Example 1, and FIG. 21 is a graph representing the changes in electromotive force occurring in the thermoelectric device plotted with respect to hydrogen concentrations when the hydrogen sensing takes place in the thermochemical gas sensor to which the single thermoelectric device composed of the BiₓTe_{y} nanowires is applied according to Example 1.

Referring to FIGS. 20 and 21, it can be known that the temperature and the electromotive force were increased as a hydrogen concentration was increased. With a hydrogen flow at 5 vol %, which is the maximum hydrogen concentration condition, the maximum electromotive force was 32.11 μV.

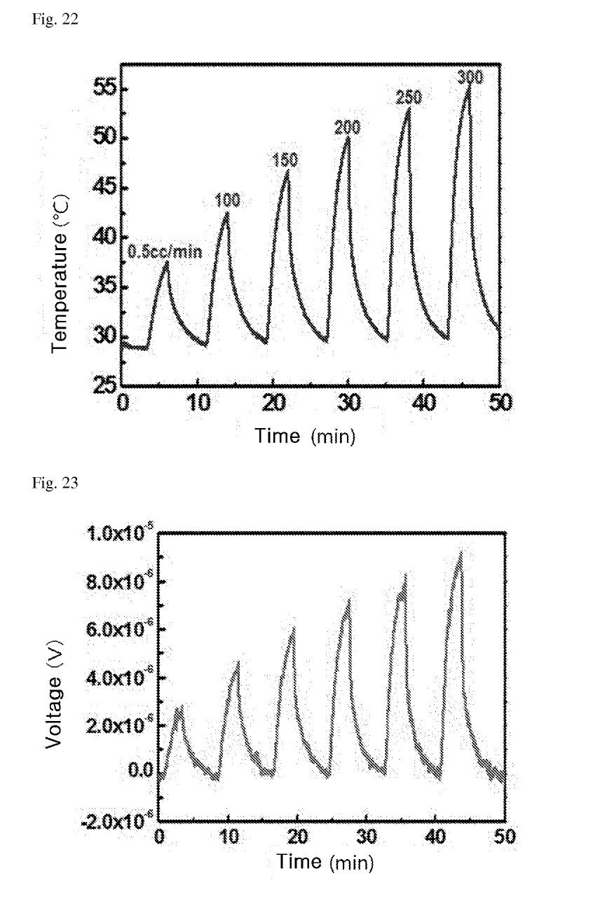

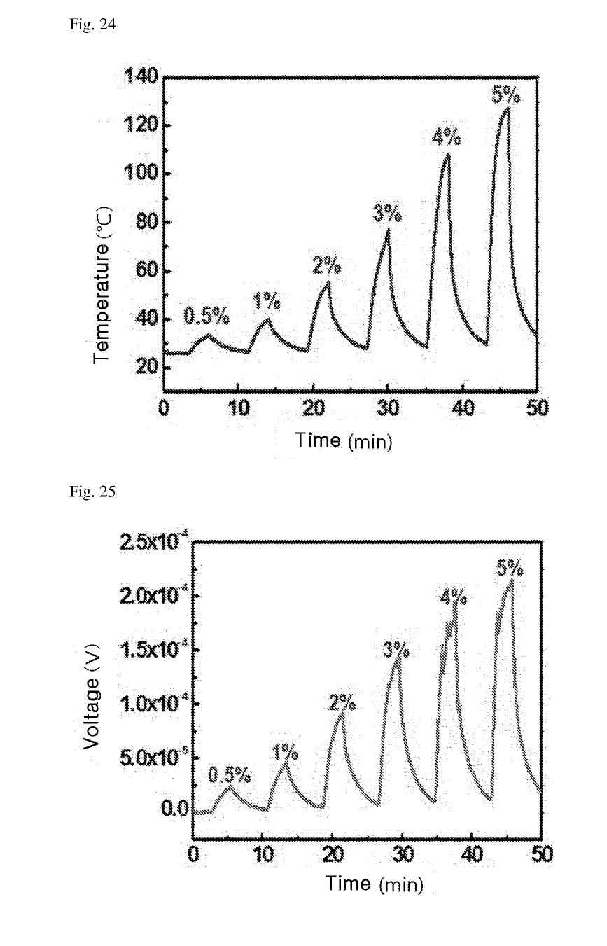

FIG. 22 is a graph representing the changes in temperature of a catalyst plotted with respect to an increase in flow rate of hydrogen under the condition in which 1 vol % hydrogen flows in the thermochemical gas sensor to which the single thermoelectric device composed of the BiₓTe_{y} nanowires is applied according to Example 1, and FIG. 23 is a graph representing the changes in electromotive force occurring in the thermoelectric device plotted with respect to the increase in flow rate of hydrogen under the condition in which 1 vol % hydrogen flows in the thermochemical gas sensor to which the single thermoelectric device composed of the BiₓTe_{y} nanowires is applied according to Example 1.

Referring to FIGS. 22 and 23, as a hydrogen flow rate increased, a temperature and an electromotive force increased. It is considered that this is because a hydrogen content increased for the same amount of time in the limited space as the flow rate increased. When the hydrogen flow rate was maximum 300 cc/min, the electromotive force was 9.2 μV.

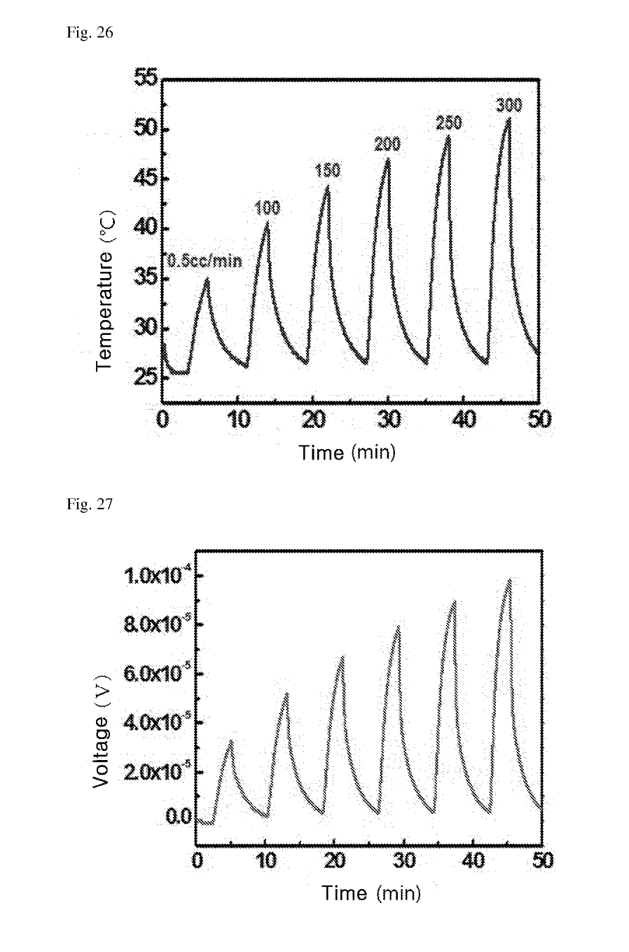

FIG. 24 is a graph representing the changes in temperature of a catalyst plotted with respect to hydrogen concentrations, when hydrogen sensing takes place in a thermochemical gas sensor to which a thermoelectric device composed of P(SbₓTe_{y})—N(BiₓTe_{y}) junction nanowires is applied according to Example 2, and FIG. 25 is a graph representing the changes in electromotive force occurring in the thermoelectric device plotted with respect to hydrogen concentrations, when the hydrogen sensing takes place in the thermochemical gas sensor to which the thermoelectric device composed of the P(SbₓTe_{y})—N(BiₓTe_{y}) junction nanowires is applied according to Example 2.

Referring to FIGS. 24 and 25, it was confirmed that a temperature and an electromotive force linearly increased by a hydrogen concentration. In this case, when maximum 5 vol % hydrogen was flown, the electromotive force was 0.215 mV, which is approximately 6 times higher than the electromotive force acquired in the single thermoelectric device. By being converted into an electromotive force per unit area, the value is approximately 17 times higher than the electromotive force acquired in the single thermoelectric device.

FIG. 26 is a graph representing the changes in temperature of a catalyst plotted with respect to an increase in flow rate of hydrogen under the condition in which 1 vol % hydrogen flows, when the hydrogen sensing takes place in the thermochemical gas sensor to which the thermoelectric device composed of the P(SbₓTe_{y})—N(BiₓTe_{y}) junction nanowires is applied according to Example 2, and FIG. 27 is a graph representing the changes in electromotive force occurring in the thermoelectric device plotted with respect to the increase in flow rate of hydrogen under the condition in which 1 vol % hydrogen flows, when the hydrogen sensing takes place in the thermochemical gas sensor to which the thermoelectric device composed of the P(SbₓTe_{y})—N(BiₓTe_{y}) junction nanowires is applied according to Example 2.

Referring to FIGS. 26 and 27, as a hydrogen flow rate increased, a temperature and an electromotive force increased, and when the hydrogen gas was flown at maximum 300 cc/min, the electromotive force was 98.3 μV, which is approximately 10 times higher than that of the single thermoelectric device, and by being converted into an electromotive force per unit area, approximately 27 times higher than that of the single thermoelectric.

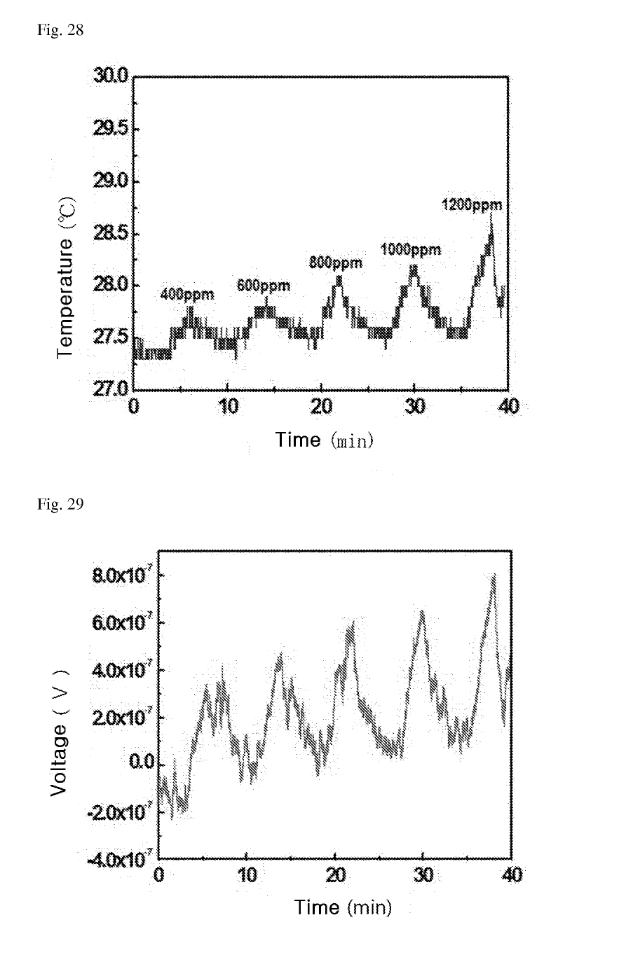

FIG. 28 is a graph representing the changes in temperature at a low concentration when the hydrogen sensing takes place in the thermochemical gas sensor to which the thermoelectric device composed of the P(SbₓTe_{y})—N(BiₓTe_{y}) junction nanowires is applied according to Example 2, and FIG. 29 is a graph representing the changes in electromotive force at a low concentration when the hydrogen sensing takes place in the thermochemical gas sensor to which the thermoelectric device composed of the P(SbₓTe_{y})—N(BiₓTe_{y}) junction nanowires is applied according to Example 2.

Referring to FIGS. 28 and 29, the change in electromotive force was observed with minimum 400 ppm (0.2 vol %) of hydrogen. However, according to the graph pattern, it is considered that the hydrogen sensing is possibly performed at an even lower hydrogen concentration.

Although exemplary embodiments of the present invention have been described in detail above, the present invention is not limited to these, and can be modified in various forms within the scope of the technical idea of the present invention by those of ordinary skill in the art.

### DESCRIPTIONS OF REFERENCE NUMERALS

10: Porous alumina template
12: Pores
20: Seed layer
30, 50, 60: Chalcogenide-based nanowires
40: Electrode

### INDUSTRIAL APPLICABILITY

A thermochemical gas sensor of the present invention can be utilized as a new type of thermoelectric nanowire array-based thermochemical gas sensor, which can sense a gas and evaluate a gas sensing property, and hence has an industrial applicability.

1. A thermochemical gas sensor, comprising:
a porous alumina template having top, bottom and side surfaces and including a plurality of pores penetrating the top and bottom surfaces;
a seed layer with electric conductivity, which is formed on the bottom surface of the porous alumina template to cover the plurality of pores;
a plurality of chalcogenide-based nanowires, which are in contact with the seed layer exposed through the plurality of pores and formed in the plurality of pores;
an electrode, which is in contact with the chalcogenide-based nanowires and formed on the top surface of the porous alumina template;
electrode wires electrically connected with the electrode; and
a porous platinum-alumina composite or porous palladium-alumina composite, which is formed above the electrode and causes an exothermic reaction when in contact with a gas to be sensed,
wherein the chalcogenide-based nanowires consist of BiₓTe_{y} (1.5≦x≦2.5, 2.4≦y≦3.6), SbₓTe_{y} (1.5≦x≦2.5, 2.4≦y≦3.6) or (Bi₁₋ₓSbₓ)Te₃ (0<x<1).

2. The thermochemical gas sensor of claim 1, wherein the seed layer has a thickness of 10 to 1000 nm, and consists of at least one metal selected from gold (Au), silver (Ag) and copper (Cu), the pores have an average diameter of 10 to 1000 nm, the chalcogenide-based nanowires have an average diameter of 1 to 500 nm which is smaller than that of the pores, the length of the chalcogenide-based nanowires is the same as or smaller than the depth of the pores, and the porous platinum-alumina composite or porous palladium-alumina composite is a porous material having a plurality of macropores and a plurality of mesopores.

3. A thermochemical gas sensor, comprising:
a porous alumina template having top, bottom and side surfaces and including a plurality of pores penetrating the top and bottom surfaces;
a seed layer with electric conductivity, which is formed on the bottom surface of the porous alumina template to cover the plurality of pores;
a plurality of P-type chalcogenide-based nanowires which are in contact with the seed layer exposed through the plurality of pores, and formed in the plurality of pores;
a plurality of N-type chalcogenide-based nanowires which are in contact with the seed layer exposed through the plurality of pores, and formed in the plurality of pores;
an electrode, which is in contact with the P-type chalcogenide-based nanowires and the N-type chalcogenide-based nanowires and formed on the top surface of the porous alumina template;
electrode wires electrically connected with the electrode; and
a porous platinum-alumina composite or porous palladium-alumina composite, which is formed above the electrode and causes an exothermic reaction when in contact with a gas to be sensed,
wherein the P-type chalcogenide-based nanowires consist of SbₓTe_{y} (1.5≦x≦2.5, 2.4≦y≦3.6) or (Bi₁₋ₓSbₓ)Te₃ (0<x<1), and
the N-type chalcogenide-based nanowires consist of BiₓTe_{y} (1.5≦x≦2.5, 2.4≦y≦3.6).

4. The thermochemical gas sensor of claim 3, wherein the seed layer has a thickness of 10 to 1000 nm, and consists of at least one metal selected from gold (Au), silver (Ag) and copper (Cu), the pores have an average diameter of 10 to 1000 nm, the chalcogenide-based nanowires have an average diameter of 1 to 500 nm which is smaller than that of the pores, the length of the chalcogenide-based nanowires is the same as or smaller than the depth of the pores, and the porous platinum-alumina composite or porous palladium-alumina composite is a porous material having a plurality of macropores and a plurality of mesopores.

5. A method of manufacturing a thermochemical gas sensor, comprising:
preparing a porous alumina template having top, bottom and side surfaces and including a plurality of pores penetrating the top and bottom surfaces, and forming a seed layer with electric conductivity on the bottom surface of the porous alumina template to cover the plurality of pores;
growing and forming a plurality of chalcogenide-based nanowires on the seed layer exposed through the plurality of pores using electrodeposition;
forming an electrode on the top surface of the porous alumina template to be in contact with the chalcogenide-based nanowires;
forming electrode wires electrically connected with the electrode; and
forming a porous platinum-alumina composite or porous palladium-alumina composite above the electrode formed on the top surface of the porous alumina template, the composite causing an exothermic reaction when in contact with a gas to be sensed,
wherein the chalcogenide-based nanowires consist of BiₓTe_{y} (1.5≦x≦2.5, 2.4≦y≦3.6), SbₓTe_{y} (1.5≦x≦2.5, 2.4≦y≦3.6) or (Bi₁₋ₓSbₓ)Te₃ (0<x<1), and
the electrodeposition uses an electrolyte containing at least one material selected from a bismuth (Bi) precursor and an antimony (Sb) precursor; a tellurium (Te) precursor; and an acid, the acid is a material that can dissolve at least one material selected from the bismuth (Bi) precursor and the antimony (Sb) precursor, and the tellurium (Te) precursor.

6. The method of claim 5, wherein the bismuth (Bi) precursor is Bi(NO₃)₃.5H₂O, the antimony (Sb) precursor is Sb₂O₃, the tellurium (Te) precursor is TeO₂, and the acid is HNO₃.

7. The method of claim 5, wherein, when the chalcogenide-based nanowires consist of SbₓTe_{y} (1.5≦x≦2.5, 2.4≦y≦3.6) or (Bi₁₋ₓSbₓ)Te₃ (0<x<1), annealing is performed on the chalcogenide-based nanowires at 100 to 300° C. prior to the formation of the electrode after the growth of the chalcogenide-based nanowires.

8. The method of claim 5, wherein the seed layer is formed to have a thickness of 10 to 1000 nm, and consists of at least one metal selected from gold (Au), silver (Ag) and copper (Cu).

9. The method of claim 5, wherein the electrode is formed by electroplating at least one metal selected from gold (Au), silver (Ag) and copper (Cu), and the electroplating is performed by applying a current to two-electrode system using a rectifier while stirring with a magnetic bar.

10. The method of claim 5, wherein the pores have an average diameter of 10 to 1000 nm,
the chalcogenide-based nanowires are formed to have an average diameter of 1 to 500 nm, which is smaller than that of the pores, and
the length of the chalcogenide-based nanowires is the same as or smaller than the depth of the pores.

11. The method of claim 5, wherein the forming of the porous platinum-alumina composite or porous palladium-alumina composite comprises:
preparing a mixed solution of styrene and distilled water;
synthesizing a polystyrene solution by adding potassium persulfate to the mixed solution;
drying the polystyrene solution to obtain colloidal crystals;
synthesizing a precursor solution of the platinum-alumina composite or palladium-alumina composite;
immersing the colloidal crystals obtained by drying in the precursor solution of the platinum-alumina composite or palladium-alumina composite; and
drying and calcining the colloidal crystals immersed in the precursor solution of the platinum-alumina composite or palladium-alumina composite to remove the polystyrene colloidal crystals,
wherein the porous platinum-alumina composite or porous palladium-alumina composite is formed to have a plurality of macropores and a plurality of mesopores.

12. A method of manufacturing a thermochemical gas sensor, comprising:
preparing a porous alumina template having top, bottom and side surfaces and including a plurality of pores penetrating the top and bottom surfaces, masking regions of the bottom surface of the porous alumina template, except the part in which chalcogenide-based nanowires are to be formed, and forming a seed layer with electric conductivity on an exposed part to cover a plurality of pores;
covering a region in which N-type chalcogenide-based nanowires are to be formed on the top surface of the porous alumina template with a first mask, and growing and forming a plurality of P-type chalcogenide-based nanowires on the seed layer exposed through the plurality of pores using electrodeposition;
covering a region in which the P-type chalcogenide-based nanowires have been formed with a second mask, and growing and forming a plurality of N-type chalcogenide-based nanowires on the seed layer exposed through the plurality of pores by removal of the first mask using electrodeposition;
forming an electrode in contact with the P-type chalcogenide-based nanowires and the N-type chalcogenide-based nanowires, on the top surface of the porous alumina template;
forming electrode wires electrically connected with the electrode; and
forming a porous platinum-alumina composite or porous palladium-alumina composite above the electrode formed on the top surface of the porous alumina template, the composite causing an exothermic reaction when in contact with a gas to be sensed,
wherein the P-type chalcogenide-based nanowires consist of SbₓTe_{y} (1.5≦x≦2.5, 2.4≦y≦3.6) or (Bi₁₋ₓSbₓ)Te₃ (0<x<1),
the N-type chalcogenide-based nanowires consist of BiₓTe_{y} (1.5≦x≦2.5, 2.4≦y≦3.6),
the electrodeposition for forming the P-type chalcogenide-based nanowires uses an electrolyte containing one or both of an antimony (Sb) precursor and a bismuth (Bi) precursor, a tellurium (Te) precursor and an acid,
the electrodeposition for forming the N-type chalcogenide-based nanowires uses an electrolyte containing a bismuth (Bi) precursor, a tellurium (Te) precursor and an acid, and
the acid is a material that can dissolve an antimony (Sb) precursor, a bismuth (Bi) precursor and a tellurium (Te) precursor.

13. The method of claim 12, wherein the bismuth (Bi) precursor is Bi(NO₃)₃.5H₂O, the antimony (Sb) precursor is Sb₂O₃, the tellurium (Te) precursor is TeO₂, and the acid is HNO₃.

14. The method of claim 12, wherein when the chalcogenide-based nanowires consist of SbₓTe_{y} (1.5≦x≦2.5, 2.4≦y≦3.6) or (Bi₁₋ₓSbₓ)Te₃ (0<x<1), annealing is performed on the chalcogenide-based nanowires at 100 to 300° C. prior to the forming of the electrode after the growth of the chalcogenide-based nanowires.

15. The method of claim 12, wherein the seed layer is formed to have a thickness of 10 to 1000 nm, and consists of at least one metal selected from gold (Au), silver (Ag) and copper (Cu).

16. The method of claim 12, wherein the electrode is formed by electroplating at least one metal selected from gold (Au), silver (Ag) and copper (Cu), and the electroplating is performed by applying a current to two-electrode system using a rectifier while stirring with a magnetic bar.

17. The method of claim 12, wherein the pores have an average diameter of 10 to 1000 nm,
the chalcogenide-based nanowires are formed to have an average diameter of 1 to 500 nm, which is smaller than that of the pores, and
the length of the chalcogenide-based nanowires is the same as or smaller than the depth of the pores.

18. The method of claim 12, wherein the forming of the porous platinum-alumina composite or porous palladium-alumina composite comprises:
preparing a mixed solution of styrene and distilled water;
synthesizing a polystyrene solution by adding potassium persulfate to the mixed solution;
drying the polystyrene solution to obtain colloidal crystals;
synthesizing a precursor solution of the platinum-alumina composite or palladium-alumina composite;
immersing the colloidal crystals obtained by drying in the precursor solution of the platinum-alumina composite or palladium-alumina composite; and
drying and calcining the colloidal crystals immersed in the precursor solution of the platinum-alumina composite or palladium-alumina composite to remove the polystyrene colloidal crystals,
wherein the porous platinum-alumina composite or porous palladium-alumina composite is formed to have a plurality of macropores and a plurality of mesopores.
